(19) United States
(12) Patent Application Publication
(10) Pub. No.: US 2026/0151464 A1
(43) Pub. Date: Jun. 4, 2026

(54) THROMBOLYTIC AGENTS FOR INTRAVASCULAR CLOTS

(71) Applicant: JINIS CO., LTD., Wanju-gun (KR)

(72) Inventors: Seong Tshool HONG, Jeonju-si (KR); Hyeon Jin KIM, Wanju-gun (KR); Mdmehedi HASSAN, Jeonju-si (KR)

(21) Appl. No.: 19/461,724

(22) Filed: Jan. 28, 2026

Related U.S. Application Data
(62) Division of application No. 17/770,795, now Pat. No. 12,599,653.

(30) Foreign Application Priority Data
Oct. 22, 2019 (KR) 10-2019-0131585

(51) Int. Cl. A61K 38/48; A61P 7/02; C12N 9/50

(52) U.S. Cl. CPC A61K 38/484; A61K 38/482; A61P 7/02; C12N 9/50; C12Y 304/21073

(57) ABSTRACT

Provided is a thrombolytic agent for ‘intravascular thrombus’, and more particularly, to a thrombolytic agent having a thrombo-recognition domain and a thrombolytic domain. It also relates to a polypeptide for thrombolysis of an intravascular thrombus, a gene for that polypeptide, and a pharmaceutical composition containing the same. The polypeptide that recognizes ‘intravascular thrombus’ and dissolves thrombus of the present invention is characterized in that it consists of a thrombolytic domain comprising the amino acid sequence set forth in SEQ ID NO: 1 or SEQ ID NO: 2 and a thrombo-recognition domain comprising the amino acid sequence set forth in SEQ ID NO: 3 or SEQ ID NO: 4. According to the present invention, the polypeptide for dissolving thrombus by recognizing ‘intravascular thrombus’ dissolves thrombus in the blood of a mammal without serious bleeding side effects has a preventive and therapeutic effect on thrombosis, thus preventing thrombosis and related diseases.

### INCORPORATION OF SEQUENCE LISTING

This application contains a sequence listing in Computer Readable Form (CRF). The CRF file containing the sequence listing entitled “8-PK3805353DIV-SeqListing.xml”, which was created and modified on Jan. 26, 2026, and is 21,976 bytes in size. The information in the sequence listing is incorporated herein by reference in entirety.

### TECHNICAL FIELD

The present invention relates to a thrombolytic agent for ‘intravascular thrombus’, and more particularly, to a thrombolytic agent having a thrombo-recognition domain and a thrombolytic domain. The present invention also relates to a polypeptide for thrombolysis of an intravascular thrombus, a gene for that polypeptide, and a pharmaceutical composition containing the same.

### BACKGROUND ART

When a blood vessel tissue is injured, blood flows out of the blood vessel. To prevent bleeding, a blood clot forms in the blood vessel tissue around the wound. This tissue thrombus, which is confined to the wound site and prevents bleeding, is a normal wound healing process and is an essential process for the survival of animals including humans. On the other hand, an abnormal blood clot within a blood vessel is referred as ‘intravascular thrombus’. If the blood clot is not removed, blood flow is blocked, causing the occurrence of thrombosis. When blood flow is blocked, it causes fatal thrombosis diseases such as stroke, pulmonary infarction, and myocardial infarction that cause tissue necrosis due to hypoxia. Therefore, if thrombosis occurs, immediate treatment is urgently needed.

Various therapeutic agents have been developed to treat thrombosis, which is a very serious disease and has a high incidence rate. Thrombolytics are direct therapeutic agents that dissolve thrombus, such as tissue-type plasminogen activator (tPA) (U.S. Pat. Nos. 4,766,075, 5,185,259, 5,587,159, 5,869,314, 6,274,335), urokinase (U.S. Pat. Nos. 4,259,447, 4,851,345, 5,055,295, 6,759,042), and streptokinase (U.S. Pat. Nos. 3,855,065, 5,011,686), U.S. Pat. No. 7,105,327). These thrombolytic agents, i.e., tPA (alteplase, reteplase), urokinase, and streptokinase, all bind to plasminogen in the body and activate it into plasmin, the activated enzyme that breaks down the thrombus, resulting thrombolysis. However, the activated thrombolytic plasmin also degrades hemostatic blood clots that were formed to prevent bleeding from damaged blood vessels, causing severe bleeding from the wound site. This is because plasmin is a non-specific thrombolytic agent that does not have specificity for ‘intravascular thrombus’. Because of the serious bleeding side effects of plasmin, the thrombolytic agents that produce plasmin, such as tPA, urokinase, and streptokinase, are all used only in limited cases.

Because of the fatal side effects of these thrombolytics, anticoagulants such as heparin, warfarin, davigatran, etc., or antiplatelets such as aspirin are used clinically instead of thrombolytic drugs for thrombotic patients. Anticoagulants and antiplatelet agents cannot dissolve clots that have already formed but prevent the additional clot formation in the blood vessels. Thus, they have no significant effect on the treatment of thrombosis although they do not cause fatal systemic bleeding, unlike plasmin-activating thrombolytics. In addition, anticoagulants and antiplatelet agents interfere with the formation of normal hemostatic blood clots and thus interfere with wound healing, resulting in serious bleeding from damaged vessels without wound healing.

Therefore, in order to treat thrombosis, there is an urgent need to develop a thrombolytic agent specific to ‘intravascular thrombus’ that accurately recognizes and decomposes only ‘intravascular thrombus’, but not hemostatic thrombus, and minimizes bleeding side effects without interfering with normal wound healing.

### DISCLOSURE OF INVENTION

### Technical Problem

The present inventors recognized that an ideal thrombosis treatment would be a thrombolytic agent specific for the ‘intravascular thrombus’ that dissolves ‘intravascular thrombus’ only without interfering with the normal blood clotting process and wound healing that occurs in vascular tissue. Efforts were made to develop a thrombolytic agent specific for the ‘intravascular thrombus’. Therefore, one object of the present invention is to provide an innovative thrombolytic agent for ‘intravascular thrombus’ as a thrombosis treatment, which does not have any serious bleeding side effects such as fatal systemic bleeding due to its specific dissolution of ‘intravascular thrombus’ causing thrombosis.

### Technical Solution

To achieve the above goal, the present invention provides a polypeptide having a thrombolytic domain comprising the amino acid sequence shown in SEQ ID NO: 1 or SEQ ID NO: 2 and a thrombo-recognition domain comprising the amino acid sequence shown in SEQ ID NO: 3 or SEQ ID NO: 4, thereby providing a polypeptide for the recognition and dissolution of ‘intravascular thrombi’.

The present invention also provides a thrombolytic domain gene, characterized in that it has the nucleotide sequence shown in SEQ ID NO: 5 or SEQ ID NO: 6, which encodes a thrombolytic domain having the amino acid sequence shown in SEQ ID NO: 1 or SEQ ID NO: 2.

The present invention also provides a thrombo-recognition domain gene encoding a thrombo-recognition domain having an amino acid sequence shown in SEQ ID NO: 3 or SEQ ID NO: 4, characterized in that it has a nucleotide sequence shown in SEQ ID NO: 7 or SEQ ID NO: 8.

The present invention also provides a pharmaceutical composition for treating or preventing thrombosis and related diseases, characterized in that it contains a polypeptide or a gene encoding the same as an active ingredient by recognizing ‘intravascular thrombus’ and dissolving the ‘intravascular thrombus’.

### Benefits

According to the present invention, the polypeptide for dissolving thrombus by recognizing ‘intravascular thrombus’ dissolves thrombus in the blood of a mammal without serious bleeding side effects, thereby having preventive and therapeutic efficacy against thrombosis.

### BRIEF DESCRIPTION OF THE DRAWINGS

FIGS. 1A-1D are results of confirming the thrombolytic ability after treatment of the thrombolytic enzyme SK or HtrA1 in the ex vivo thrombus according to Experimental Example 1 of the present invention (FIG. 1A; image in which the thrombus is dissolved (HA1) and undissolved thrombus mass (SK), FIG. 1B; thrombus solubility expressed in % before treatment, FIG. 1C; fibrin degradation products (FDP) from thrombotic dissolution, FIG. 1D; the amount of D-dimer from thrombotic dissolution (Ctrl: control, SK: streptokinase, HA1: Polypeptide HtrA1 that recognizes ‘intravascular thrombus’ and dissolves thrombus)).

FIGS. 2A-2D are results of confirming the thrombolytic ability after treatment of the thrombolytic enzyme SK or HtrA2 in the ex vivo thrombus according to Experimental Example 1 of the present invention (FIG. 2A; image in which the thrombus is dissolved (HA2) and undissolved thrombus mass (SK), FIG. 2B; thrombus solubility expressed in % before treatment, FIG. 2C; fibrin degradation products (FDP) from thrombotic dissolution, d; the amount of D-dimer from thrombotic dissolution, (Ctrl: control, SK: streptokinase, HA2: Polypeptide HtrA2 that recognizes ‘intravascular thrombus’ and dissolves thrombus)).

FIGS. 3A and 3B are results of confirming the activity of plasminogen activation to plasmin of the ‘intravascular thrombolytic’ polypeptide HtrA1 according to Experimental Example 2 of the present invention (FIG. 3A; measurement of fluorescence intensity of a substrate dissolved by plasmin generated by plasminogen activation; FIG. 3B; SDS-PAGE image confirming the plasmin band generated by the activation of plasminogen (Ctrl: control, PL: plasmin, SK: streptokinase, UK: urokinase, HA1: Polypeptide HtrA1 that recognizes ‘intravascular thrombus’ and dissolves thrombus)).

FIGS. 4A and 4B are results of confirming the activity of plasminogen activation to plasmin of the ‘intravascular thrombolytic’ polypeptide HtrA1 according to Experimental Example 2 of the present invention (FIG. 4A; measurement of fluorescence intensity of a substrate dissolved by plasmin generated by plasminogen activation; FIG. 4B; SDS-PAGE image confirming the plasmin band generated by the activation of plasminogen (Ctrl: control, PL: plasmin, SK: streptokinase, UK: urokinase, HA2: Polypeptide HtrA2 that recognizes ‘intravascular thrombus’ and dissolves thrombus)).

FIGS. 5A-5C are results of confirming the activity of the intravascular thrombolytic polypeptide of the present invention on the fibrinolysis components of the wound healing process in order to evaluate whether the thrombolytic polypeptide has thrombus specificity according to Experimental Example 3 of the present invention (FIG. 5A; SDS-PAGE image to confirm the degradation of fibrinogen into fibrin by treatment with each testing thrombolytic enzyme, FIG. 5B; Immuno-blot image to confirm the degradation of cellular fibronectin by treatment with each testing thrombolytic enzyme, FIG. 5C; Immuno-blot image to confirm the degradation of plasma fibronectin by treatment with each testing thrombolytic enzyme (Ctrl: control, PL: plasmin, SK: streptokinase, UK: urokinase, HA1: Polypeptide HtrA1 that recognizes ‘intravascular thrombus’ and dissolves thrombus)).

FIG. 6 is a thrombosis tail image result confirming the therapeutic efficacy of the ‘intravascular thrombolytic’ polypeptide of the present invention on thrombosis in tail thrombosis mice using an animal model according to Example 1 of the present invention (Ctrl: control, PL: Plasmin, SK: streptokinase, UK: urokinase, tPA: tissue plasminogen activator, HA1: Polypeptide HtrA1 that recognizes ‘intravascular thrombus’ and dissolves thrombus, HA2: Polypeptide HtrA2 that recognizes ‘intravascular thrombus’ and dissolves thrombus).

FIG. 7 is an H&E staining image result of thrombotic tail tissue confirming the therapeutic efficacy of the ‘intravascular thrombolytic’ polypeptide of the present invention on thrombosis in tail thrombosis mice using an animal model according to Example 1 of the present invention (Ctrl: control, PL: Plasmin, SK: streptokinase, UK: urokinase, tPA: tissue plasminogen activator, HA1: Polypeptide HtrA1 that recognizes ‘intravascular thrombus’ and dissolves thrombus, HA2: Polypeptide HtrA2 that recognizes ‘intravascular thrombus’ and dissolves thrombus).

FIG. 8 is a bleeding image result confirming the effect of the ‘intravascular thrombolytic’ polypeptide of the present invention on wound healing using a wound animal model according to Example 3 of the present invention (Ctrl: control, PL: Plasmin, SK: streptokinase, UK: urokinase, tPA: tissue plasminogen activator, HA1: Polypeptide HtrA1 that recognizes ‘intravascular thrombus’ and dissolves thrombus, HA2: Polypeptide HtrA2 that recognizes ‘intravascular thrombus’ and dissolves thrombus).

FIGS. 9A-9D are bleeding test results confirming the effect of the ‘intravascular thrombolytic’ polypeptide HtrA1 of the present invention on wound healing using a wound animal model according to Example 3 of the present invention. (FIG. 9A; Measurement of bleeding time, FIG. 9B; Measurement of hemorrhage, FIG. 9C; Measurement of hemoglobin content in the bleeding fluid, FIG. 9D; Measurement of time taken to wound healing and blood clotting (Ctrl: control, PL: plasmin, SK: streptokinase, UK: urokinase, HA1: Polypeptide HtrA1 that recognizes ‘intravascular thrombus’ and dissolves thrombus)).

FIGS. 10A-10D are bleeding test results confirming the effect of the ‘intravascular thrombolytic’ polypeptide HtrA1 of the present invention on wound healing using a wound animal model according to Example 3 of the present invention. (FIG. 10A; Measurement of bleeding time, FIG. 10B; Measurement of hemorrhage, FIG. 10C; Measurement of hemoglobin content in the bleeding fluid, FIG. 10D; Measurement of time taken to wound healing and blood clotting (Ctrl: control, PL: plasmin, SK: streptokinase, UK: urokinase, HA2: Polypeptide HtrA2 that recognizes ‘intravascular thrombus’ and dissolves thrombus)).

### DETAILED DESCRIPTION OF PREFERRED EMBODIMENTS

Various agents have been developed to dissolve thrombus, an intravascular blood clot, but all the conventional thrombolytic agents have serious bleeding side effects, making it difficult to effectively treat thrombosis and related diseases. Furthermore, these treatments cannot effectively prevent thrombosis.

The present inventors focused on the fact that ‘intravascular thrombus’ is a kind of protein aggregate and that quality control proteins that degrade the aggregated proteins are essential for the survival of organisms. Through experiments were carried out to identify an endogenous proteinase having a domain that recognizes misfolded/aggregated protein and dissolves misfolded/aggregated protein in the body, and to confirm that this endogenous proteinase can dissolve the aggregated blood clots in blood vessels.

Accordingly, in the present invention, an extensive search was made for domains that recognize misfolded/aggregated proteins, for domains that dissolve misfolded/aggregated proteins, and for quality control proteins including these domains. As a result, it was confirmed that a polypeptide having a thrombo-recognition domain and a thrombolytic domain, can treat thrombosis without serious bleeding side effects by specifically recognizing ‘intravascular thrombus’ and specifically dissolving ‘intravascular thrombus’.

In the present invention, a polypeptide that recognizes and dissolve ‘intravascular thrombus’ with an intravascular thrombo-recognition domain and a thrombolytic domain and dissolves a thrombus was confirmed that (1) it had excellent thrombolytic activity, (2) unlike conventional thrombolytic agents, there was no risk of bleeding due to plasmin production since it does not activate plasminogen into plasmin, (3) unlike conventional thrombolytic agents, it did not break down proteins which are important for wound healing, such as fibrinogen, c-fibronectin, and p-fibronectin, so that it does not interfere with wound healing, (4) it effectively treated thrombosis in an in vivo animal model, (5) it completely treated thromboembolism with a 100% survival rate in an in vivo animal model, unlike conventional thrombolytic agents, (6) there were no bleeding side effects as it did not interfere with blood clotting and wound healing of in vivo bleeding animal experiments, unlike conventional thrombolytic agents.

In one embodiment of the present invention, with the therapeutic efficacy in the mouse tail thrombosis model, it was confirmed that the polypeptide that recognizes and dissolves ‘intravascular thrombus’ of the present invention has cured thrombosis, unlike conventional thrombolytic agents.

In another embodiment of the present invention, it was confirmed that the polypeptide that recognizes and dissolves ‘intravascular thrombus’ of the present invention has cured fatal thromboembolism with a 100% survival rate while conventional thrombolytic agents failed to treat but resulted in deaths of mice.

In addition, in the mouse tail bleeding experiment, the polypeptide that recognizes and dissolves ‘intravascular thrombus’ of the present invention was confirmed to have a perfect wound healing efficacy without any bleeding side effects, being indistinguishable to the untreated control group in measures such as bleeding time at the wound site, amount of bleeding, amount of hemoglobin lost, and blood clotting time.

Accordingly, in one aspect, the present invention relates to a polypeptide that recognizes ‘intravascular thrombus’ and dissolves thrombus, wherein said polypeptide is characterized in that it consists of a thrombolytic domain comprising the amino acid sequence set forth in SEQ ID NO: 1 or SEQ ID NO: 2 and a thrombo-recognition domain comprising the amino acid sequence set forth in SEQ ID NO: 3 or SEQ ID NO: 4.

In the present invention, said polypeptide is composed of a thrombolytic domain comprising the amino acid sequence set forth in SEQ ID NO: 1 and a thrombo-recognition domain comprising the amino acid sequence set forth in SEQ ID NO: 3, or said polypeptide is composed of a thrombolytic domain comprising the amino acid sequence set forth in SEQ ID NO: 2 and a thrombo-recognition domain comprising the amino acid sequence set forth in SEQ ID NO: 4.

In the present invention, said polypeptide that recognizes the ‘intravascular thrombus’ and dissolves the thrombus is characterized in that it belongs to the serine protease which is a trypsin-like polypeptide, preferably High Temperature Requirement (Htr) family. More preferably, it is characterized in that it contains HtrA1 and HtrA2.

Said thrombolytic domain may be a domain having 50% or more, preferably 80% or more, more preferably 90% or more homology to the amino acid sequence of SEQ ID NO: 1 or SEQ ID NO: 2, wherein the thrombolytic domain is characterized as a domain conferring serine protease activity, including a peptide.

Said thrombo-recognition domain may be a domain having 50% or more, preferably 80% or more, more preferably 90% or more homology to the amino acid sequence of SEQ ID NO: 3 or SEQ ID NO: 4 and is characterized in that it has a topology structure composed of multiple combinations of beta-strand and alpha-helix. Thus, said thrombo-recognition domain may be a PDZ domain or a PDZ-like domain having a function of recognizing ‘intravascular thrombus’ and regulating the activity of a thrombolytic enzyme.

In another aspect, the present invention relates to a gene for the thrombolytic domain comprising the nucleotide sequence shown in SEQ ID NO: 5 or SEQ ID NO: 6, which encodes a thrombolytic domain having the amino acid sequence shown in SEQ ID NO: 1 or SEQ ID NO: 2.

Said thrombolytic domain gene is characterized in that it has 50% or more, preferably 80% or more, more preferably 90% or more homology to the nucleotide sequence of SEQ ID NO: 5 or SEQ ID NO: 6.

In another aspect, the present invention relates to a gene for the thrombo-recognition domain comprising the nucleotide sequence shown in SEQ ID NO: 3 or SEQ ID NO: 4, which encodes a thrombo-recognition having the amino acid sequence shown in SEQ ID NO: 7 or SEQ ID NO: 8.

Said thrombo-recognition domain gene is characterized in that it has 50% or more, preferably 80% or more, more preferably 90% or more homology to the nucleotide sequence of SEQ ID NO: 7 or SEQ ID NO: 8.

In another aspect, the present invention relates to a pharmaceutical composition for treating or preventing thrombosis and related diseases, characterized in that it contains a polypeptide or a gene encoding the same as an active ingredient for recognizing ‘intravascular thrombus’ and dissolving the thrombus.

The pharmaceutical composition of the present invention may include a pharmaceutically acceptable carrier commonly used in formulation, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc. can be exemplified, The present invention is not limited thereto.

The pharmaceutical composition of the present invention may further include a lubricant, a wetting agent, a sweetening agent, a flavoring agent, an emulsifying agent, a suspending agent, a preservative, and the like, in addition to the above components. Suitable pharmaceutically acceptable carriers and agents are described in detail in Remington's Pharmaceutical Sciences (19th ed., 1995).

The pharmaceutical composition of the present invention may be administered orally or parenterally, preferably parenterally, and in the case of parenteral administration, intramuscular injection, intravenous injection, subcutaneous injection, intraperitoneal injection, topical administration, transdermal administration, etc.

A suitable dosage of the pharmaceutical composition of the present invention is variously prescribed depending on factors such as formulation method, administration method, age, weight, sex, pathological condition, food, administration time, administration route, excretion rate, and reaction sensitivity of the patient. Meanwhile, the preferred dosage of the pharmaceutical composition of the present invention is 0.0001-1000 μg per day.

The pharmaceutical composition of the present invention is prepared in unit dosage form by formulating using a pharmaceutically acceptable carrier and/or excipient according to a method that can be easily carried out by a person of ordinary skill in the art to which the present invention pertains. Or it can be prepared by introducing into a multi-dose container. In this case, the formulation may be in the form of a solution, suspension, or emulsion in oil or aqueous medium, or in the form of an extract, powder, granule, tablet, or capsule, and may additionally include a dispersant or stabilizer.

The pharmaceutical composition for treating or preventing thrombosis and related diseases, according to the present invention, has the effect of dissolving ‘intravascular thrombus’ while minimizing bleeding side effects when administered to a mammal.

In the present invention, the thrombosis and related diseases can be selected from the group that includes, but is not limited to, Thrombosis, Embolism, Thromboembolism, Arterial Thromboembolism, Venous Thromboembolism (VTE), cardiovascular disease, cerebrovascular disease, ischemic disease, etc. Specifically, it can be selected from the group that includes, but is not limited to, Deep Vein Thrombosis (DVT), Pulmonary Embolism (PE), ischemic stroke, stroke, cerebral hemorrhage, cerebral infarction, myocardial infarction, heart attack, and angina (unstable angina).

### EXAMPLES

Hereinafter, the present invention will be described in more detail through specific examples. However, these Examples are only for describing the present invention in more detail, and the scope of the present invention is not limited by these Examples.

### Experiment 1: Evaluation of Ex Vivo Thrombolytic Efficacy

Polypeptides HtrA1 and HtrA2 that recognize ‘intravascular thrombus’ and dissolve thrombus were prepared as recombinant proteins. The cDNA corresponding to 157-480 of the HtrA1 amino acid sequence (SEQ ID NO: 9) containing both the intravascular thrombo-recognition domain and the thrombolytic domain was used with a forward primer (5′-AATTCATATGCAAGGGCAGGAAGATCCCA-3′) (SEQ ID NO: 11) and a reverse primer (5′-TATCTCGAGCTATGGGTCAATTTCTTCGGG-3′) (SEQ ID NO: 12) for amplification by PCR. For PCR conditions, after initial denaturation at 95° C. for 5 minutes, amplification (30 seconds at 95° C., 1 minute at 63° C., 2 minutes at 72° C.) was repeated 34 times, followed by 10 minutes at 72° C. The amplified product was subcloned into the NdeI/XhoI site of the expression vector pET28a+ (Novagen, USA). The obtained HtrA1 recombinant plasmid was electroporated into E. coli BL21 (DE3) pLysS (Stratagene, USA) and cultured with addition of IPTG for expression of HtrA1. The expressed culture medium was sonicated to make cell lysate, passed through econo-pac chromatography column (Bio-Rad), and then purified by PD-10 column (Amersham, US) to obtain recombinant protein HtrA1. In the case of HtrA2, the cDNA corresponding to 134-458 of the HtrA2 amino acid sequence (SEQ ID NO: 10) was used with a forward primer (5′-GTCCTCGCCCATATGGCCGTCCCTAGCC-3′) (SEQ ID NO: 13) and a reverse primer (5′-GGCTCTCGAGTCATTCTGTGACCTCAGGG-3′) (SEQ ID NO: 14) was amplified by PCR. For PCR, after initial denaturation at 95° C. for 5 minutes, amplification (30 seconds at 95° C., 45 seconds at 65° C., 1 minute at 72° C.) was repeated 34 times, followed by 10 minutes at 72° C. After obtaining this, the recombinant protein HtrA2 was obtained by subcloning and expressing the pET28a+ expression vector (Novagen, USA) in the same manner as for HtrA1.

The thrombolytic activity of the recombinant proteins, HtrA1 and HtrA2, was confirmed with ex vivo thrombus. A thrombus was prepared using platelet-rich blood, and either 50 mM Tris-HCl control or each thrombolytic enzyme (2 mg/ml) was treated to the thrombus at 37° C. for 24 hours. After treatment, the weight of the thrombus was measured and expressed as a percentage before treatment. In addition, quantification was made for fibrin degradation products (FDP) and D-dimer generated by the degradation of fibrin polymer constituting the thrombus.

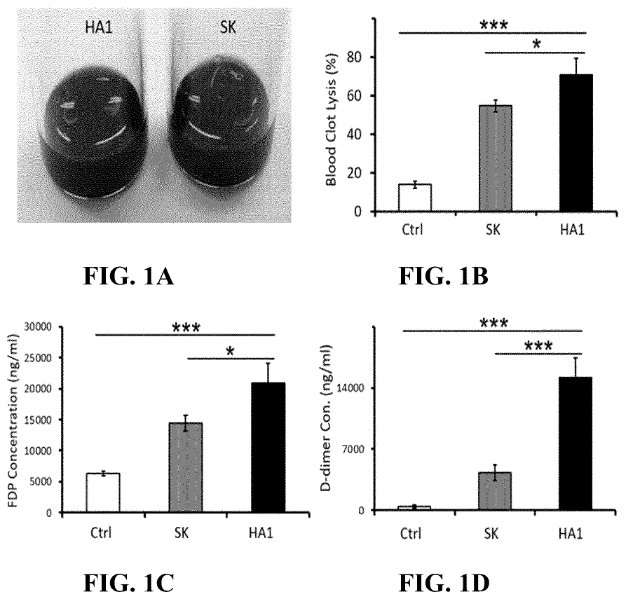

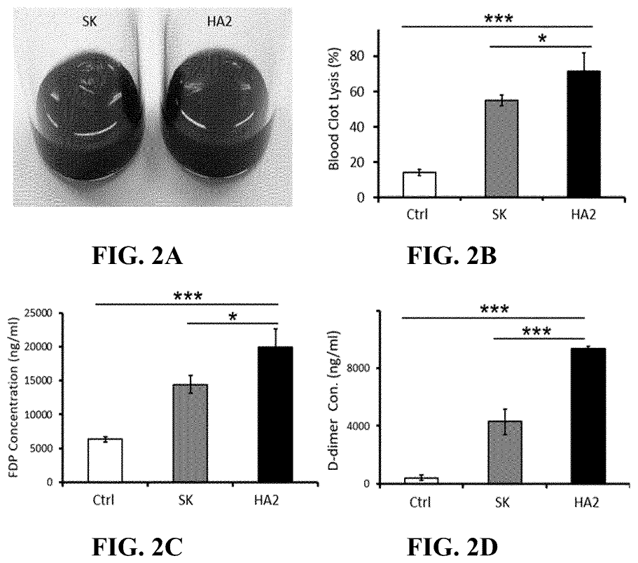

In FIGS. 1A-1D, the thrombolytic ability of HtrA1 (FIGS. 1A, 1B) and fibrin degradation ability (FIGS. 1C-1D) were the most excellent compared with the conventional thrombolytic enzymes. In FIGS. 2A-2D, HtrA2 also showed superior thrombolytic activity (FIGS. 2A-2B) and fibrin degrading ability (FIGS. 2C-2D) than conventional thrombolytic enzymes.

### Experiment 2: Evaluation of Plasmin Generation (Thrombolytic Mechanism)

The current thrombolytic mechanism of thrombolytic agent is plasmin-dependent fibrinolysis by activating plasmininogen into plasmin. To confirm the mechanism of action compared to the conventional thrombolytic enzymes, each thrombolytic enzyme (0.1 mg/ml) was added to plasminogen (1.23 μM) and plasmin-specific fluorescent substrate, Boc-Glu-Lys-Lys-MCA (100 μM). After incubation, the activation of plasminogen to plasmin was confirmed by measuring the fluorescence intensity of the substrate dissolved by plasmin. Plasmin bands generated by the actual activation of plasminogen to plasmin were confirmed by SDS-PAGE after each enzyme (0.15 mg/ml) was added to plasminogen (5.14 μM).

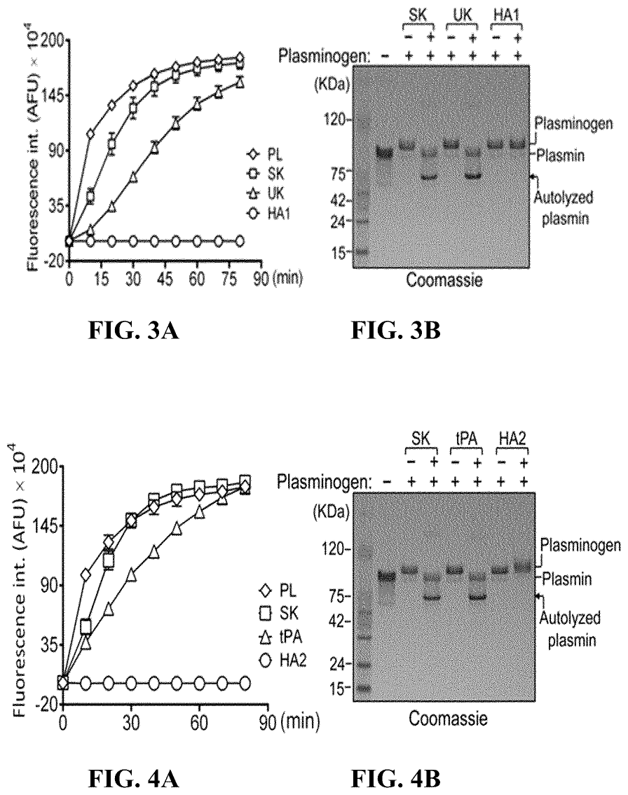

Compared with the conventional thrombolytic enzyme, HtrA1 did not activate plasminogen (FIG. 3A), and thus there was no plasmin production (FIG. 3B). Similarly, HtrA2 also had no effect on plasminogen (FIG. 4A), and as a result, there was no plasmin production by HtrA2 (FIG. 4B).

### Experiment 3: Evaluation of Thrombus Specificity

To evaluate the thrombus specificity of HtrA1, the fibrinolytic activities of components involved in the wound healing process were investigated. The fibrin clot obtained by reacting fibrinogen with thrombin was incubated with 50 mM Tris-HCl control or thrombolytic enzyme (2 mg/ml) at 37° C. for 24 hours. Dissolution of the fibrin clot was measured at a wavelength of 415 nm using a spectrophotometer. To see the activity of HtrA1 on fibrinogen, 5 μM fibrinogen was incubated with 50 mM Tris-HCl control or each thrombolytic enzyme (0.15 mg/ml) for 3 hours at 37° C. SDS-PAGE was carried out to confirm the fibrinolytic activity of HtrA1 on fibrinogen. To see the activity of HtrA1 on fibronectin, 1.5 μM fibronectin was incubated with 50 mM Tris-HCl control or each thrombolytic enzyme (0.15 mg/ml) for 3 hours at 37° C. and separated on a 4-12% SDS gel for immunostaining with cFN or anti-pFN antibody.

As shown in Table 1, HtrA1 and HtrA2 not only showed a statistically significant difference in absorbance when compared to the untreated control group, but also showed a statistically significant difference in absorbance for the conventional thrombolytic enzyme streptokinase. For fibrin clot dissolution, HtrA1 was the best, followed by HtrA2.

TABLE 1

| Groups | A415 |
|---|---|
| Control | 1.870 ± 0.05 |
| Streptokinase | 1.335 ± 0.07^{a} |
| Urokinase | 1.047 ± 0.06^{a} |
| HtrA1 | 1.055 ± 0.10^{a,b} |
| HtrA2 | 1.002 ± 0.09^{a,b} |

^{a}p < 0.05 significance difference from control group
^{b}p < 0.01 significance difference from Streptokinase group

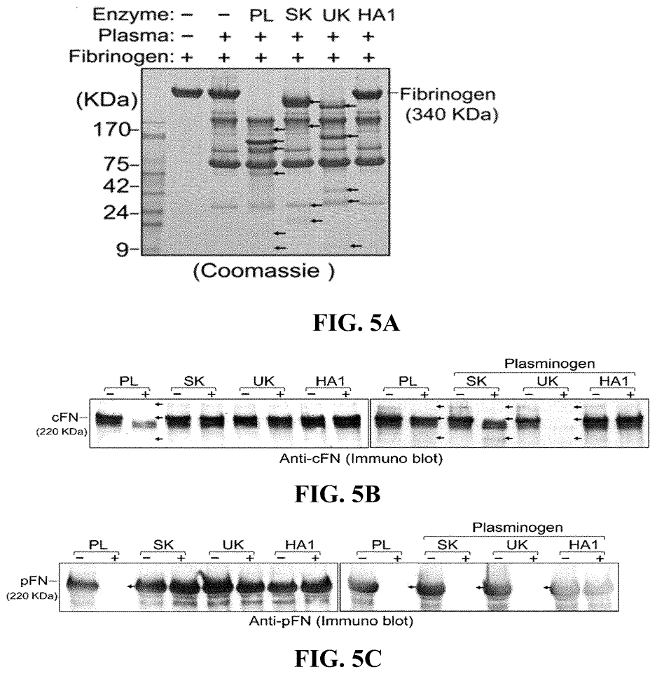

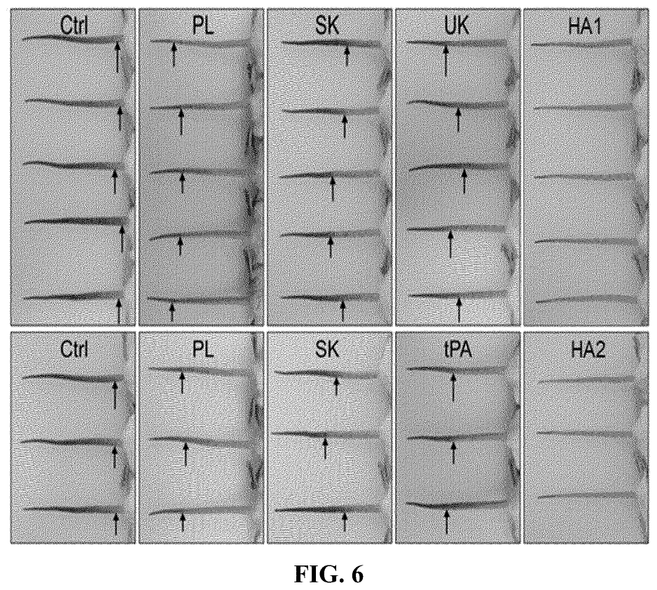

Unlike conventional thrombolytic enzymes, HtrA1 did not degrade fibrinogen, a critical component for hemostasis at the wound site (FIG. 5A). In addition, HtrA1 did degrade neither cellular fibronectin (FIG. 5B) nor plasma fibronectin (FIG. 5C).

Finally, to examine the activity of HtrA1 on the wound healing process, an external incised wound (˜30 mm2) was made from the tail skin of BALB/c mice. The excised wound pieces were incubated with 50 mM Tris-HCl control or each thrombolytic enzyme (2 mg/ml) at 37° C. for 72 hours. Wound healing was determined by observing the liquid containing the wound pieces and measuring the absorbance at 550 nm. In this in vivo wound tissue experiment using animals, treatment with the conventional thrombolytic enzyme resulted in continued bleeding due to the interruption of wound healing process while normal wound healing occurred in the treatment group of HtrA1 or HtrA2 (Table 2).

TABLE 2

| Groups | A550 |
|---|---|
| Control | 0.235 ± 0.031 |
| Streptokinase | 0.471 ± 0.018^{a} |
| Urokinase | 0.404 ± 0.037^{a} |
| HtrA1 | 0.203 ± 0.028^{a,b} |
| HtrA2 | 0.247 ± 0.033^{a,b} |

^{a}p < 0.05 significance difference from control group
^{b}p < 0.01 significance difference from Streptokinase group

### Example 1: Therapeutic Efficacy for Treatment of Thrombosis

An animal experiment using a tail thrombosis model was performed to evaluate the thrombosis treatment efficacy of the thrombolytic agents of the present invention. A K-carrageenan-induced tail thrombosis model was established in 15-week-old female BALB/c mice. Saline (control) or each thrombolytic enzyme was intraperitoneally injected into each group (n=8) of mice with tail thrombosis, and the length and proportion of the thrombosis site were measured 24 hours later, and the results are shown in Tables 3 and 4.

TABLE 3

| | | Tail | Thrombosis | Thrombosis |
|---|---|---|---|---|
| | Dose | Length | Length | Ratio |
| Groups | (mg/kg) | (cm) | (cm) | (%) |
| Control | — | 9.21 ± 0.08 | 9.07 ± 0.11^{a} | 98.50 ± 0.80^{a} |
| Plasmin | 40 | 9.02 ± 0.07 | 3.37 ± 0.45^{b} | 37.39 ± 5.10^{b} |
| Streptokinase | 40 | 9.10 ± 0.10 | 5.11 ± 1.22^{a} | 56.16 ± 13.4^{a} |
| Urokinase | 40 | 9.07 ± 0.10 | 4.27 ± 0.60^{a} | 47.08 ± 6.51^{a} |
| HtrA1 (10 mg/kg) | 10 | 9.18 ± 0.16 | 4.72 ± 1.06 | 51.33 ± 10.9 |
| HtrA1 (20 mg/kg) | 20 | 9.12 ± 0.10 | 3.41 ± 0.57 | 37.41 ± 6.43 |
| HtrA1 (40 mg/kg) | 40 | 9.17 ± 0.12 | 1.61 ± 0.49 | 17.56 ± 5.47 |

^{a}p < 0.001 significance difference from HtrA1 (40 mg/kg) group
^{b}p < 0.01 significance difference from HtrA1 (40 mg/kg) group

As shown in Table 3, the length and frequency of thrombosis tails in the HtrA1-treated group were significantly reduced compared to the conventional thrombolytic enzyme-treated groups in the mouse tail thrombosis model. It was shown that the thrombolytic effect has increased in proportion to the dosage. This confirms that HtrA1 has a statistically significant level of thrombosis treatment efficacy compared to conventional thrombolytic enzymes.

TABLE 4

| | | Tail | Thrombosis | Thrombosis |
|---|---|---|---|---|
| | Dose | Length | Length | Ratio |
| Groups | (mg/kg) | (cm) | (cm) | (%) |
| Control | — | 9.19 ± 0.09 | 9.03 ± 0.14^{a} | 98.25 ± 1.16^{a} |
| Plasmin | 40 | 9.03 ± 0.06 | 3.40 ± 0.41^{b} | 37.65 ± 4.62^{b} |
| Streptokinase | 40 | 9.09 ± 0.09 | 4.97 ± 1.16^{a} | 54.66 ± 12.7^{a} |
| tPA | 40 | 9.06 ± 0.08 | 3.49 ± 0.53^{b} | 38.53 ± 6.06^{b} |
| HtrA2 | 10 | 9.15 ± 0.12 | 4.55 ± 1.11 | 49.62 ± 11.5 |
| (10 mg/kg) | | | | |
| HtrA2 | 20 | 9.11 ± 0.11 | 4.01 ± 0.83 | 44.01 ± 9.18 |
| (20 mg/kg) | | | | |
| HtrA2 | 40 | 9.14 ± 0.08 | 2.19 ± 0.75 | 23.93 ± 8.20 |
| (40 mg/kg) | | | | |

^{a}p < 0.001 significance difference from HtrA2 (40 mg/kg) group
^{b}p < 0.01 significance difference from HtrA2 (40 mg/kg) group

As shown in Table 4, the length and frequency of thrombosis tails in the HtrA2-treated group were also significantly reduced compared to the conventional thrombolytic enzyme-treated groups in the mouse tail thrombosis model. It was shown that the thrombolytic effect has increased in proportion to the dosage. This confirms that HtrA2 has a statistically significant level of thrombosis treatment efficacy compared to conventional thrombolytic enzymes.

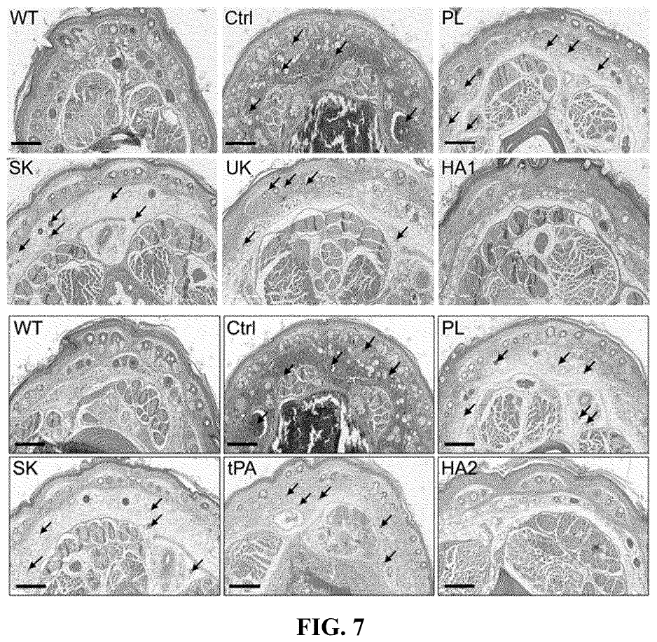

In FIG. 7, observation of the thrombosis tail tissue after H & E staining, revealed the thrombotic mass and the thrombus dissolution in the samples from the group treated with conventional thrombolytic enzymes while there was no thrombotic mass in the HtrA1 and HtrA2 treatment groups, confirming excellent thrombolytic efficacy of HtrA1 and HtrA2.

### Example 2: Therapeutic Efficacy for Treatment of Pulmonary Thromboembolism

Next, an animal experiment using a pulmonary thromboembolism model was performed to evaluate the prevention and treatment efficacy of thromboembolism. A model of pulmonary thromboembolism induced by adenosine 5′-diphosphate (ADP) was established in 15-week-old female C57BL/6 mice. Each group (n=8) of pulmonary thromboembolism mice fasted for 12 hours or longer, and then saline (control) or thrombolytic enzyme was injected intravenously at a dose of 40 mg/kg. After 30 minutes, ADP (150 mg/kg) was injected into the mice to induce pulmonary thromboembolism, and then death was confirmed, and the results are shown in Tables 5 and 6.

TABLE 5

| | Dose | | Protection rate |
|---|---|---|---|
| Groups | (mg/kg) | Lethal number/total | (%) |
| Control | — | 6/6 | 0 |
| Plasmin | 40 | 3/6 | 50 |
| Streptokinase | 40 | 4/6 | 33 |
| Urokinase | 40 | 2/6 | 67 |
| HtrA1 | 40 | 0/6 | 100 |

From Table 5, in the lung thromboembolism mouse model, the survival rates of the plasmin, streptokinase, and urokinase treatment groups were 50%, 33%, and 66%, respectively, but the survival rate of the HtrA1 treatment group was 100%. This confirms that HtrA1 in the present invention has the perfect therapeutic and preventive effect on fatal pulmonary thromboembolism.

TABLE 6

| | Dose | | Protection rate |
|---|---|---|---|
| Groups | (mg/kg) | Lethal number/total | (%) |
| Control | — | 5/5 | 0 |
| Plasmin | 40 | 2/5 | 60 |
| Streptokinase | 40 | 4/5 | 20 |
| tPA | 40 | 3/5 | 40 |
| HtrA2 | 40 | 0/5 | 100 |

From Table 6, in the pulmonary thromboembolism mouse model, the survival rates of the plasmin, streptokinase, and tPA treatment groups were 60%, 20%, and 40%, respectively, but the survival rate of the HtrA2 treatment group was 100%. This also confirms that HtrA2 has a perfect therapeutic and preventive effect on pulmonary thromboembolism.

### Example 3: Effect on Removing the Risk of Internal Bleeding

A tail bleeding test was performed using 15-week-old C57BL/6 female mice to determine whether there is a risk of internal bleeding during the wound healing process when the thrombolytic protein HtrA1 is administered. Saline (control) or each thrombolytic enzyme was intravenously injected into a group of mice (n=5) at a dose of 40 mg/kg. After 30 minutes, the tail was cut from the anesthetized mouse, and the bleeding time and amount of blood were recorded while collecting blood until the bleeding stopped, and the hemoglobin content of the collected blood was measured. In addition, the time required for blood to clot in the severed tail vein of the rat was recorded.

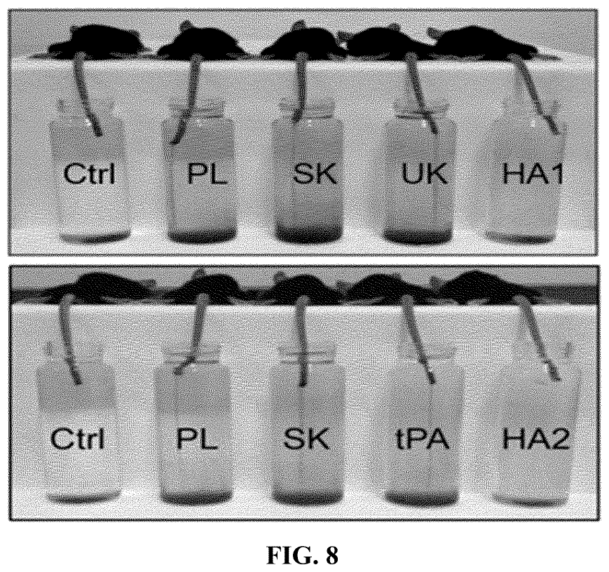

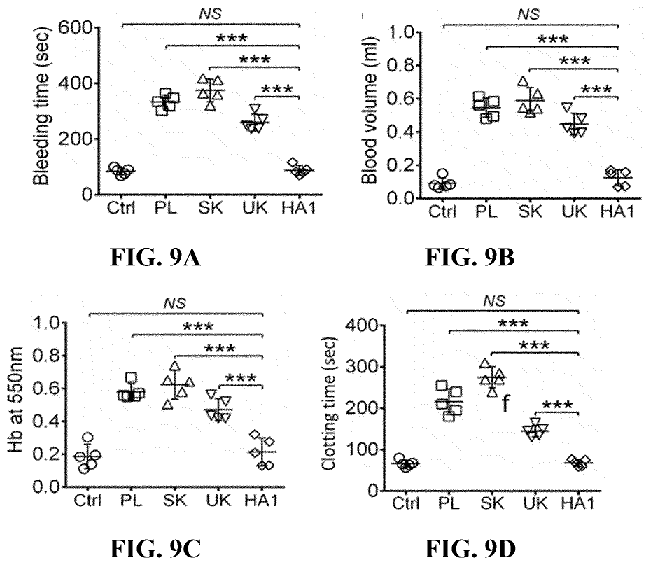

As shown in FIG. 8, it was confirmed that the bleeding of HtrA1 and HtrA2 was significantly less than that of other thrombolytic enzymes in the mouse tail bleeding experiment. In the case of the HtrA1-treated group, the actual bleeding time (FIG. 9A), the amount of bleeding (FIG. 9B), the amount of hemoglobin lost (FIG. 9C), and the time taken to hemostasis (FIG. 9D) were measured. It was confirmed that the bleeding side effect was reduced to a level similar to that of the control group, being incomparably different from those of the conventional thrombolytic enzymes.

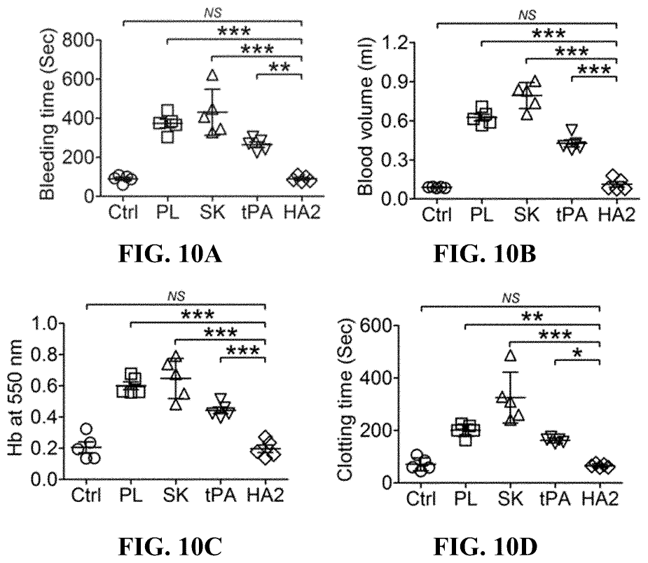

In the case of the HtrA2-treated group, the bleeding time (FIG. 10A), the amount of bleeding (FIG. 10B), the amount of hemoglobin lost (FIG. 10C), and the time taken to hemostasis (FIG. 10D) were measured. It was confirmed that the bleeding side effects were reduced to the level as much as that of the control group, being incomparably different from those of the conventional thrombolytic enzymes. HtrA1 and HtrA2 do not interfere with the hemostasis process, which is a part of the wound healing process, and thus completely reduce the risk of bleeding.

### Commercial Use Potential

According to the present invention, the polypeptide for dissolving thrombus by recognizing ‘intravascular thrombus’ dissolves thrombus in the blood of a mammal without serious bleeding side effects and has a preventive and therapeutic effect on thrombosis, thus preventing thrombosis and related diseases or it can be widely used as a therapeutic agent.

1. A pharmaceutical composition for treating thrombosis, comprising a recombinant polypeptide consisting of amino acid residues 134 to 458 of the amino acid sequence set forth in SEQ ID NO: 10.


SEQUENCE LISTING (14 sequences)

SEQ ID NO: 1 - protein, 161 aa, synthetic construct
  REGION 1..161: note = amino acid sequence of thrombolytic domain of HtrA1 (HtrA1 as1)
GSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFL

SEQ ID NO: 2 - protein, 177 aa, synthetic construct
  REGION 1..177: note = amino acid sequence of thrombolytic domain of HtrA2 (HtrA2 as1)
ILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFL

SEQ ID NO: 3 - protein, 103 aa, synthetic construct
  REGION 1..103: note = amino acid sequence of thrombus cognitive domain of HtrA1 (HtrA1 as2)
TESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNE

SEQ ID NO: 4 - protein, 82 aa, synthetic construct
  REGION 1..82: note = amino acid sequence of thrombus cognitive domain of HtrA2 (HtrA2 as2)
VMMLTLSPSILAELQLREPSFPDVQHGVLIHKVILGSPAHRAGLRPGDVILAIGEQMVQNAEDVYEAVRTQSQLAVQIRRGR

SEQ ID NO: 5 - DNA, 483 bp, synthetic construct
  misc_feature 1..483: note = base sequence of thrombolytic domain of HtrA1 (HtrA1 bs1)
  source 1..483: mol_type = other DNA
gggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctc

SEQ ID NO: 6 - DNA, 531 bp, synthetic construct
  misc_feature 1..531: note = base sequence of thrombolytic domain of HtrA2 (HtrA2 bs1)
  source 1..531: mol_type = other DNA
atcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctg

SEQ ID NO: 7 - DNA, 309 bp, synthetic construct
  misc_feature 1..309: note = base sequence of thrombus cognitive domain of HtrA1 (HtrA1 bs2)
  source 1..309: mol_type = other DNA
acggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaa

SEQ ID NO: 8 - DNA, 246 bp, synthetic construct
  misc_feature 1..246: note = base sequence of thrombus cognitive domain of HtrA2 (HtrA2 bs2)
  source 1..246: mol_type = other DNA
gtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacga

SEQ ID NO: 9 - protein, 480 aa, synthetic construct
  REGION 1..480: note = amino acid sequence of HtrA1 (HtrA1)
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 10 - protein, 458 aa, synthetic construct
  REGION 1..458: note = amino acid sequence of HtrA2 (HtrA2)
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLAAVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 11 - DNA, 29 bp, synthetic construct
  misc_feature 1..29: note = HtrA1 forward primer
  source 1..29: mol_type = other DNA
aattcatatgcaagggcaggaagatccca

SEQ ID NO: 12 - DNA, 30 bp, synthetic construct
  misc_feature 1..30: note = HtrA1 reverse primer
  source 1..30: mol_type = other DNA
tatctcgagctatgggtcaatttcttcggg

SEQ ID NO: 13 - DNA, 28 bp, synthetic construct
  misc_feature 1..28: note = HtrA2 forward primer
  source 1..28: mol_type = other DNA
gtcctcgcccatatggccgtccctagcc

SEQ ID NO: 14 - DNA, 29 bp, synthetic construct
  misc_feature 1..29: note = HtrA2 reverse primer
  source 1..29: mol_type = other DNA
ggctctcgagtcattctgtgacctcaggg